(19) United States
(12) Patent Application Publication
(10) Pub. No.: US 2024/0183835 A1
(43) Pub. Date: Jun. 6, 2024

(54) SYSTEMS AND METHODS FOR MONITORING DISC CONDITIONS OF AGRICULTURAL IMPLEMENTS

(71) Applicant: CNH Industrial Canada, Ltd., Saskatoon (CA)

(72) Inventor: James W. Henry, Saskatoon (CA)

(21) Appl. No.: 18/073,231

(22) Filed: Dec. 1, 2022

(51) Int. Cl. G01N 33/24; A01B 7/00; A01B 76/00; B23Q 17/00

(52) U.S. Cl. CPC G01N 33/24; A01B 7/00; A01B 76/00; B23Q 17/006; G01N 2033/245

(57) ABSTRACT

In one aspect, a system for monitoring the condition of discs of agricultural implements includes a plurality of discs configured to be supported relative to an agricultural implement, and a field profile sensor configured to generate data indicative of a field profile of an aft portion of the field located rearward of the plurality of discs relative to a direction of travel of the agricultural implement. In addition, the system includes a controller communicatively coupled to the field profile sensor. The controller is configured to monitor the data received from the field profile sensor and determine an operating condition of one or more of the plurality of discs based at least in part on the field profile of the aft portion of the field.

### FIELD OF THE INVENTION

The present subject matter relates generally to agricultural implements and, more particularly, to systems and methods for monitoring an operating condition of discs of an agricultural implement (e.g., a damaged or missing condition)

### BACKGROUND OF THE INVENTION

It is well known that, to attain the best agricultural performance from a piece of land, a farmer must cultivate the soil, typically through a tillage operation. Common tillage operations include plowing, harrowing, and sub-soiling. Modern farmers perform these tillage operations by pulling a tillage implement behind an agricultural work vehicle, such as a tractor. Depending on the crop selection and the soil conditions, a farmer may need to perform several tillage operations at different times over a crop cycle to properly cultivate the land to suit the crop choice.

In some configurations, a tillage implement includes a plurality of disc blades or “discs” supported on its frame. In this respect, as the tillage implement travels across the field to perform a tillage operation thereon, the discs rotate relative to the soil to cut and size the residue present on the field surface for improved microbial degradation and incorporation into the soil.

During such tillage operations, the discs may contact rocks or other buried obstacles. In certain instances, the contact between a disc and an obstacle may be sufficient to bend, break, or otherwise damage the disc. A damaged disc may, in turn, negatively impact the quality of the tillage operation being performed and should be replaced as soon as possible. Unfortunately, it can be difficult for an operator to notice a damaged disc during a tillage operation as the frame and/or the wheels of the tillage implement and/or the associated work vehicle may block the operator's view of the discs. Similarly, in instances in which a disc has fallen off the implement or is otherwise missing, it can be difficult for the operator to notice such missing disc during the performance of an agricultural operation.

Accordingly, a system and method for monitoring the operating condition of discs of an agricultural implement, such as a damaged or missing condition, would be welcomed in the technology.

### BRIEF DESCRIPTION OF THE INVENTION

Aspects and advantages of the invention will be set forth in part in the following description, or may be obvious from the description, or may be learned through practice of the invention.

In one aspect, the present subject matter is directed to a system for monitoring the condition of discs of agricultural implements. The system includes a plurality of discs configured to be supported relative to an agricultural implement, and a field profile sensor configured to generate data indicative of a field profile of an aft portion of the field located rearward of the plurality of discs relative to a direction of travel of the agricultural implement. In addition, the system includes a controller communicatively coupled to the field profile sensor. The controller is configured to monitor the data received from the field profile sensor and determine an operating condition of one or more of the plurality of discs based at least in part on the field profile of the aft portion of the field.

In another aspect, the present subject matter is directed to a method for monitoring the condition of discs of agricultural implements. The method includes receiving, with a computing device, data indicative of a field profile of an aft portion of a field located rearward of a plurality of discs of an agricultural implement relative to a direction of travel of the agricultural implement. The method also includes analyzing, with the computing device, the field profile of the aft portion of the field to determine an operating condition of one or more of the plurality of discs, and initiating, with the computing device, a control action based on the determined operating condition of one or more of the plurality of discs.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 illustrates a top view of one embodiment of an agricultural implement in accordance with aspects of the present subject matter;

FIG. 2 illustrates an exemplary baseline field profile that is created by a plurality of discs of an agricultural implement during normal operating conditions;

FIG. 3 illustrates a series of successive field profiles detected within a field during the performance of an agricultural operation, particularly illustrating examples of field profiles that are indicative of a damaged condition and a missing condition for respective discs;

FIG. 4 illustrates a schematic view of one embodiment of a system for monitoring the operating condition of discs of agricultural implements in accordance with aspects of the present subject matter; and

FIG. 5 illustrates a flow diagram of one embodiment of a method for monitoring the operating condition of discs of agricultural implements in accordance with aspects of the present subject matter.

Repeat use of reference characters in the present specification and drawings is intended to represent the same or analogous features or elements of the present technology.

### DETAILED DESCRIPTION OF THE INVENTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

In general, the present subject matter is directed to systems and methods for monitoring the operating condition of discs of an agricultural implement. Specifically, in several embodiments, the disclosed system may monitor the field profile (e.g., a surface profile or a seedbed profile) of the field behind the implement as the implement performs an agricultural operation to determine when the operating condition of a disc has changed (e.g., when the disc has become damaged or is missing). For instance, in accordance with aspects of the present subject matter, a field profile sensor (e.g., a surface profile sensor or a seedbed profile sensor) may be provided in association with the implement, with the field profile sensor being configured to capture data indicative of a profile or contour of the field rearward of the implement. When a disc is damaged or missing, the field profile of the aft portion of the field located rearward of the respective disc will differ from an expected or baseline field profile. Accordingly, a controller of the disclosed system may be configured to determine the operating condition of discs of the tillage implement based on the detected field profile of the field. Additionally, in some embodiments, the controller may further be configured to automatically initiate a control action in response to determining the operating condition of one or more discs. For instance, in one embodiment, the control action may include providing an operator notification or adjusting the operation of one or both of the tillage implement and/or the work vehicle towing the implement (e.g., stopping operation of the vehicle/implement). In addition, or as an alternative thereto, the control action may be associated with identifying the location of at which a disc was damaged or went missing, such as by mapping or georeferencing the location at which it was initially detected that the disc was damaged or missing.

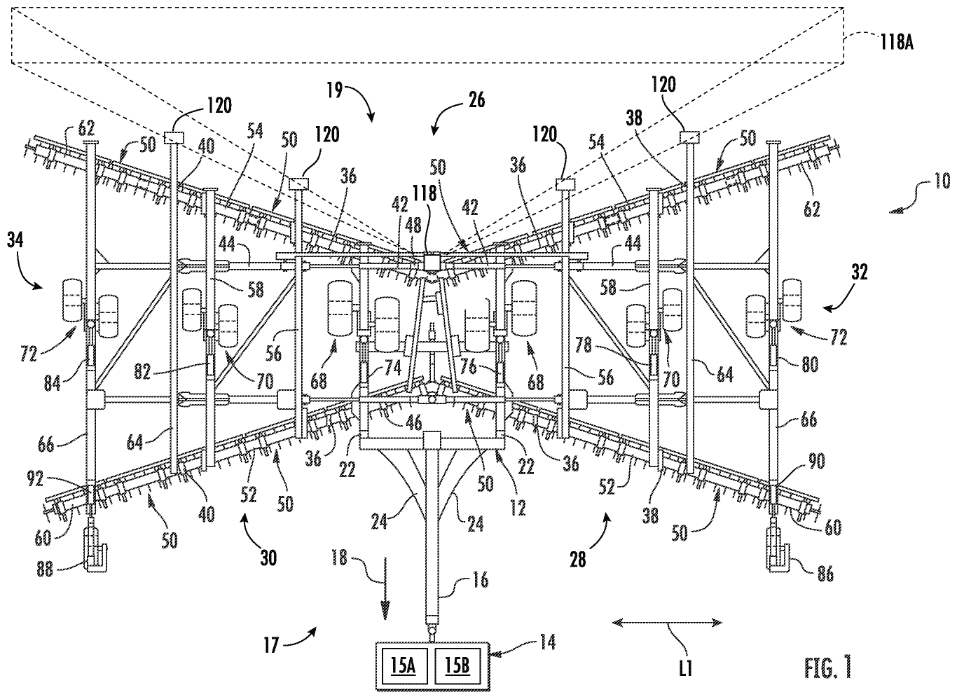

Referring now to FIG. 1, a top view of one embodiment of an agricultural implement 10 is illustrated in accordance with aspects of the present subject matter. As shown, the implement 10 is configured as a multi-wing disk ripper. However, in other embodiments, the implement 10 may have any other suitable implement configuration, such as by being configured as any other suitable tillage implement (e.g., a cultivator) or other implement (e.g., a planter, seeder, and/or the like).

As shown, the implement 10 includes a carriage frame assembly 12 configured to be towed by a work vehicle 14 (shown schematically in FIG. 1), such as a tractor. The carriage frame assembly 12 may generally extend between a forward end 17 and an aft end 19 along a forward direction of travel 18 of the implement and may include a pull hitch 16 extending in the direction of travel 18 of the implement 10 at the forward end 17 of the implement 10 and carrier frame members 22 which are coupled with and extend from the pull hitch 18. Reinforcing gusset plates 24 may be used to strengthen the connection between the pull hitch 18 and the carrier frame members 22. As shown schematically in FIG. 1, the work vehicle 14 may include an engine 15A and a transmission 15B. The transmission 15B may be operably coupled to the engine 15A and may provide variably adjusted gear ratios for transferring engine power to wheels or track assemblies (not shown) of the work vehicle 14 via a drive axle assembly (not shown) (or via axles if multiple drive axles are employed) for driving the work vehicle 14.

As shown in FIG. 1, the tillage implement 10 is configured as a multi-section implement including a plurality of frame sections spanning across the implement's lateral width in a lateral direction L1 of the implement 10. Specifically, in the illustrated embodiment, the tillage implement 10 includes a central frame section 26, inner right and left wing frame sections 28, 30 pivotally coupled to the central frame section 26, and right and left outer-wing sections 32, 34 pivotally coupled to the respective right and left inner-wing sections 28, 30. For example, each of the inner-wing sections 28, 30 is pivotally coupled to the central frame section 26 at pivot joints 36. Similarly, the right outer-wing section 32 is pivotally coupled to the right inner-wing section 28 at pivot joints 38 while the left outer-wing section 34 is pivotally coupled to the left inner-wing section 30 at pivot joints 40. As is generally understood, the pivot joints 36, 38, 40 may be configured to allow relative pivotal motion between adjacent frame sections of the implement 10. For example, the pivot joints 36, 38, 40 may allow for articulation of the various frame sections between a fully-extended position, in which the frame sections are all intended to be disposed substantially in a common plane, and a transport position, in which the wing sections 28, 30, 32, 34 are folded upwardly to reduce the overall width of the implement 10.

Additionally, as shown in FIG. 1, the implement 10 may include inner-wing actuators 42 coupled between the central frame section 26 and the inner-wing sections 28, 30 to enable pivoting or folding between the fully-extended and transport positions. For example, by retracting/extending the inner-wing actuators 42, the inner-wing sections 28, 30 may be pivoted or folded relative to the central frame section 26 about the pivot joints 36. Moreover, the implement 10 may also include outer-wing actuators 44 coupled between each inner-wing section 28, 30 and its adjacent outer-wing section 32, 34. As such, by retracting/extending the outer-wing actuators 44, each outer-wing section 32, 34 may be pivoted or folded relative to its respective inner-wing section 28, 30.

Moreover, each of the frame sections may be configured to support a plurality of ground-engaging tools, such as one or more gangs of discs 50. In such an embodiment, the gangs of discs 50 may be supported relative to frame members 46, 48, 52, 54, 60, 62 of the frame sections in any suitable manner so as to provide smooth working of the soil. It should be appreciated that, in other embodiments, any other suitable ground-engaging tools, such as shanks, tines, rolling baskets, and/or the like, may also be supported by the various frame members.

In several embodiments, the various frame sections 26, 28, 30, 32, 34 of the tillage implement 10 may be configured to be positioned at variable positions relative to the soil in order to set the position of the gangs of discs 50 above the soil as well as the penetration depth of the discs 50. For example, in the illustrated embodiment, the tillage implement 10 includes center transport wheels 68 pivotally interconnected with the carrier frames 22 so that they provide support to the forward and aft frame members 46 and 48 relative to the soil. Similarly, inner-wing transport wheels 70 may be interconnected with the frame elements 58 to support and variably position the inner-wing sections 28, 30 relative to the soil. In addition, outer-wing transport wheels 72 may be pivotally mounted on the frame members 66 to support and variably position the outer-wing sections 32, 34 relative to the soil.

In such an embodiment, wheel actuators may also be provided in operative association with the various wheels to adjust the relative positioning between the frame sections and the soil. For instance, center wheel actuators 74, 76 may be utilized to manipulate the center transport wheels 68 to establish the distance of the central frame section 26 relative to the soil while inner-wing wheel actuators 78, 82 may be used to variably position the inner-wing sections 28, 30 relative to the soil. Similarly, outer-wing wheel actuators 80, 84 may be used to variably position the outer-wing sections 32, 34 relative to the soil.

It should be appreciated that the implement 10 may also include gauge wheels 86, 88 on the outer-wing sections 32, 34 to orient the fore-to-aft angle of the tillage implement 10 relative to the soil. In such an embodiment, gauge wheel actuators 90, 92 may be provided in operative association with the gauge wheels 86, 88 to allow the fore-to-aft angle of the implement 10 to be adjusted. As shown in FIG. 1, in one embodiment, the gauge wheels 86, 88 may correspond to the forward-most ground-engaging components of the implement 10.

In accordance with aspects of the present subject matter, the implement 10 may be configured to support one or more field profile sensors 118, 120 that generate or provide data indicative of a field profile of an aft portion of the field disposed rearward of the implement 10 relative to the direction of travel 18 of the implement 10. As used herein, the term field profile may include, for example, a surface profile of the surface of the field and/or a seedbed profile of the seedbed underlying the surface of the field.

In several embodiments, the field profile sensor(s) 118, 120 may correspond to one or more surface profile sensors 118. For instance, each surface profile sensor 118 may be mounted to or supported on the implement 10, with the surface profile sensor 118 having a field of view 118A directed towards the field. Specifically, as shown in FIG. 1, each surface profile sensor 118 may be supported relative to the implement 10 (e.g., adjacent to the aft end 19 of the implement 10) such that the field of view 118A of the sensor 118 is directed towards an aft portion of the field disposed rearward of the implement 10 relative to the direction of travel 18 of the implement 10. As such, each surface profile sensor 118 may be configured to generate data indicative of the surface profile or contour of the portion of the field located behind or aft of the implement 10. In this regard, each surface profile sensor 118 may be configured as any suitable device, such as a LIDAR device(s), camera(s) (e.g., a stereo or 3-D camera(s)), radar sensor(s), ultrasonic sensor(s), and/or the like, that allows the sensor 118 to generate point-cloud data, image data, radar data, ultrasound data, and/or the like indicative of the surface profile of the aft portion of the field.

It should be appreciated that, while the implement 10 is shown as only including or being associated with one surface profile sensor 118, the implement 10 may include or be associated with any other suitable number of surface profile sensors 118, such as two or more surface profile sensors 118. For instance, in one embodiment, the number of soil profile sensors 118 may be selected such that the sensor(s) collectively has(have) a field of view that extends across the width of the implement 10 in the lateral direction L1 and, thus, allows surface profile data to be collected at every field location positioned aft of a given disc 50 of the implement 10. Further, in alternative embodiments, the surface profile sensor(s) 118 may be supported at any other suitable location on the implement 10 and/or the work vehicle 14 towing the implement 10 such that the field of view 118A of the sensor 118 is directed towards the aft portion of the field behind the implement 10.

Moreover, in addition to the surface profile sensor(s) 118 (or as an alternative thereto), the field profile sensor(s) 118, 120 may correspond to one or more seedbed profile sensors 120. In general, each seedbed profile sensor 120 may be mounted to or supported on the implement 10, with the seedbed profile sensor 120 having a field of view (not shown) directed towards the field. Specifically, in accordance with aspects of the present subject matter, each seedbed profile sensor 120 may be supported relative to the implement 10 (e.g., adjacent to the aft end 19 of the implement 10) such that a field of view of the sensor 120 is directed towards an aft portion of the field disposed rearward of the implement 10 relative to the direction of travel 18 of the implement 10. As such, each seedbed profile sensor 120 may be configured to generate data indicative of the seedbed profile or contour of the portion of the field located behind or aft of the implement 10. In this regard, each seedbed profile sensor 120 may be configured as any suitable device that allows the sensor 120 to generate data indicative of the profile of the seedbed located aft of the implement 10. For instance, in several embodiments, each seedbed profile sensor 120 may be configured as a ground-penetrating radar (GPR) device configured to transmit waves that penetrate through the soil surface and reflect at least partially off the seedbed floor located below the soil surface to allow the GPR device to collect data indicative of the profile or contour of the seedbed. In another embodiment, each seedbed profile sensor 120 may be configured as an electromagnetic induction (EMI) device.

It should be appreciated that, while the implement 10 is shown as including or being associated with four seedbed profile sensors 120, the implement 10 may include or be associated with any other suitable number of seedbed profile sensors 120, such as three or fewer seedbed profile sensors 120 or five or more seedbed profile sensors 120. For instance, in one embodiment, the number of seedbed profile sensors 120 may be selected such that the sensor(s) collectively has(have) a field of view that extends across the width of the implement 10 in the lateral direction L1 and, thus, allows seedbed profile data to be collected at every field location positioned aft of a given disc 50 of the implement 10. Further, in alternative embodiments, the seedbed profile sensor(s) 120 may be supported at any other suitable location on the implement 10 and/or the work vehicle 14 towing the implement 10 such that the field of view of the sensor(s) 120 is directed towards the aft portion of the field behind the implement 10.

It should also be appreciated that the configuration of the implement 10 described above and shown in FIG. 1 is provided only to place the present subject matter in an exemplary field of use. Thus, it should be appreciated that the present subject matter may be readily adaptable to any manner of implement configuration.

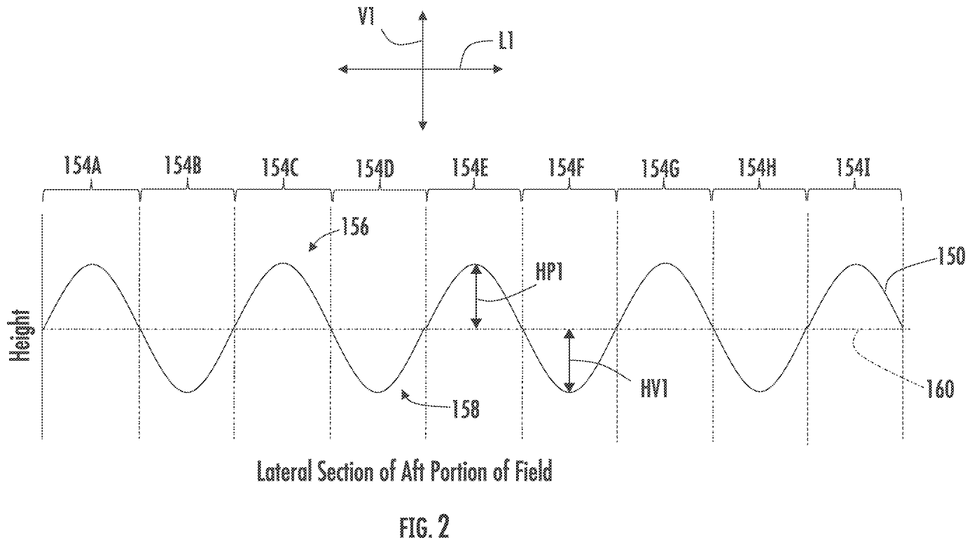

During normal operating conditions for the discs 50 (e.g., when the discs 50 are not damaged or missing), the discs 50 will generally work the soil such that a known or expected field profile will be located immediately behind the implement 10. Specifically, under normal operating conditions, the working of the soil by the discs 50 generally results in both a surface profile across the field surface and a seedbed profile across the seedbed located beneath the field surface that has uniform pattern. For instance, FIG. 2 illustrates an exemplary field profile (which could represent a surface profile of the field and/or a seedbed profile of the field) created by a number of discs 50 during normal operating conditions. As shown, the field profile (indicated by solid line 150) generally has a uniform pattern (e.g., sinusoidal pattern) defined relative to a plane extending along the lateral direction L1 of the implement 10 and a vertical direction V1 of the implement 10, with the vertical direction V1 extending perpendicular to both the direction of travel 18 and the lateral direction L1 of the implement 10. This uniform pattern across the lateral width of the implement 10 generally defines a baseline field profile (e.g., a baseline surface profile or a baseline seedbed profile) during normal operating conditions of the discs 50.

As shown in FIG. 2, in several embodiments, the field profile 150 created by the discs 50 may be divided along the lateral direction L1 into lateral portions or “lanes” 154, with each lane 154 corresponding to a lateral portion of the field aligned with and worked by a respective disc 50 of the implement 10. For example, in the illustrated field profile, a first lane 154A is aligned with a first one of the discs 50 in the direction of travel 18 of the implement, a second lane 154B is aligned with a second one of the discs 50 in the direction of travel 18 of the implement, a third lane 154C is aligned with a third one of the discs 50 in the direction of travel 18 of the implement, a fourth lane 154D is aligned with a fourth one of the discs 50 in the direction of travel 18 of the implement, a fifth lane 154E is aligned with a fifth one of the discs 50 in the direction of travel 18 of the implement, a sixth lane 154F is aligned with a sixth one of the discs 50 in the direction of travel 18 of the implement, a seventh lane 154G is aligned with a seventh one of the discs 18 in the direction of travel 18 of the implement, an eighth lane 154H is aligned with a eighth one of the discs 50 in the direction of travel 18 of the implement, and a ninth lane 154I is aligned with a ninth one of the discs 50 in the direction of travel 18 of the implement, with the field profile within each lane 154 being affected by the operating condition of the respective disc 50. Given the sinusoidal pattern created by the discs 50, each lane 154 is generally characterized by a peak 156 or a valley 158 of the field profile 150.

As shown in FIG. 2, during normal operating conditions, the baseline field profile generally defines a uniform shape or dimensions along the sinusoidal pattern created by the discs 50. For example, each peak 156 along the field profile 150 generally defines a baseline height HP1 relative to a nominal or average height reference (indicated by dashed line 160). Similarly, each valley 158 along the field profile 150 generally defines a baseline height HV1 relative to the nominal height reference 160.

Based on the field profile defined across the different lanes 154A-154I, the operating condition of each respective disc 50 may be determined. For example, the measured field profile provided by the field profile sensors 118, 120 (e.g., the measured surface profile provided by the surface profile sensor(s) 118 and/or the measured seedbed profile provided by the seedbed profile sensor(s) 120) may be compared to the baseline field profile 150 illustrated in FIG. 2 to determine when the operating condition of one of the discs 50 has changed, such as by comparing the shape or dimensions of the baseline field profile 150 to the shape or dimensions of the measured field profile provided by the field profile sensor(s) 118, 120. Specifically, in several embodiments, the baseline heights HP1, HV1 for the peaks/valleys 156, 160 may be indicative of normal operating conditions of the discs 50. Accordingly, any deviation or repeated deviation from the baseline heights HP1, HV1 may be indicative of a change in the operating condition of a given disc(s) 50.

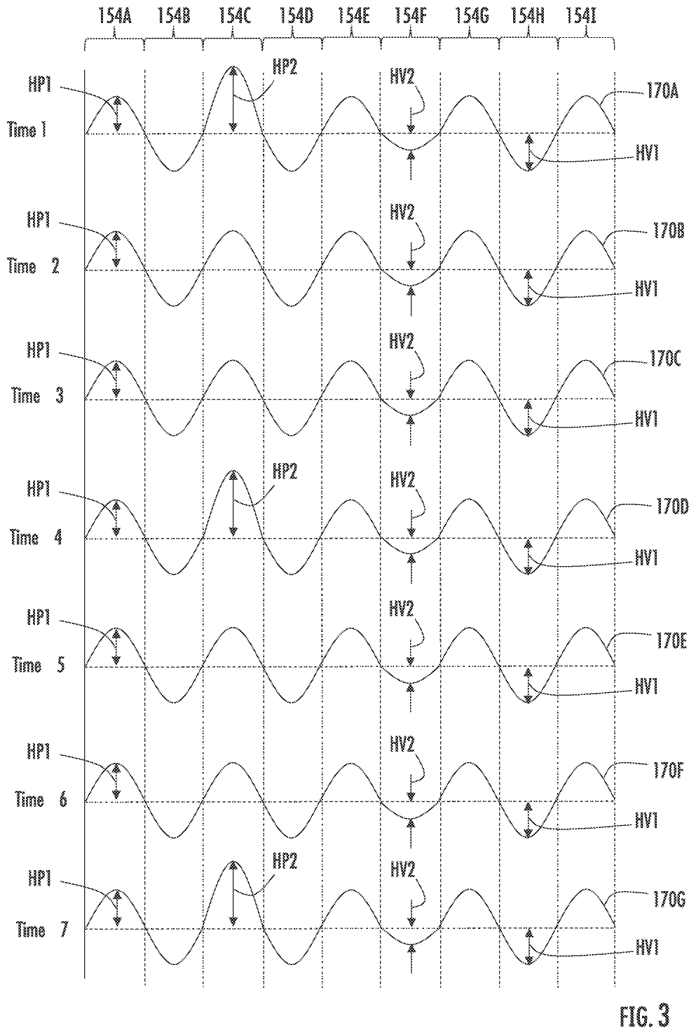

For example, FIG. 3 illustrates a series of successive field profiles 170 (e.g., field profiles 170A-170G) measured by a given field profile sensor(s) 118, 120 (e.g., either the surface profile sensor(s) 118 or the seedbed profile sensor(s) 120) along a lateral width of the implement 10 encompassing the various lanes 154A-154I described above, with each measured field profile 170 being captured at a given time (e.g., Time 1, Time 2, Time 3, Time 4, Time 5, Time 6, Time 7) extending across a time period from Time 1 to Time 7. As shown, the field profile 170 defined within each of lanes 154A, 154B, 154D, 154E, 154G, 154H, and 154I generally matches the baseline field profile 150 described above with reference to FIG. 2 across the entire time period. For instance, within each of lanes 154A, 154E, 154G, and 154I, a peak is defined within each measured field profile 170 that generally has a height that matches the associated baseline height HP1 of the baseline field profile 150. Similarly, within each of lanes 154B, 154D, and 154H, a valley is defined within each measured field profile 170 that generally has a height that matches the associated baseline height HV1 of the baseline field profile 150.

However, within lane 154C, the height of the associated peak varies across the time period. Specifically, referring to field profiles 170A, 170D, and 170G (i.e., at Times 1, 4, and 7), the peak within lane 154C defines a height HP2 that exceeds the associated baseline height HP1 of the baseline field profile. However, referring to field profiles 170B, 170C, 170E, and 170F (i.e., at Times 2, 3, 5, and 6), the peak within lane 154C defines a height that that generally matches the associated baseline height HP1 of the baseline field profile 150. Such a cyclical deviation from the baseline field profile 150 is generally indicative of a damaged condition of the associated disc 50, such as when the disc 50 has become bent or broken. In particular, with a bent or broken disc 50, the field profile will deviate from the baseline field profile 150 as the bent or broken portion of the disc 50 rotates through the soil, but will return back to the baseline field profile 150 as the remainder of the disc 50 (i.e., the remaining portion that is not bent or broken) rotates through the soil. Thus, by recognizing this cyclical deviation from the baseline field profile 150, it may be determined or inferred that the disc 50 aligned with lane 154C is damaged or otherwise has a damaged condition.

Similarly, referring to the field profiles 170 defined within lane 154F, the height of the associated valley consistently or uniformly varies from the baseline field profile 150 across the entire time period. Specifically, within each measured field profile 170, the valley within lane 154F defines a height HV2 that is significantly less than the associated baseline height HV1 of the baseline field profile 150. Such a constant deviation from the baseline field profile 150 is generally indicative of a missing condition of the associated disc 50, such as when the disc 50 has fallen off the implement 10. In particular, with a missing disc 50, the field profile will consistently deviate from the baseline field profile 150 since no disc exists within the associated lane to work the soil. Thus, by recognizing this constant deviation from the baseline field profile 150, it may be determined or inferred that the disc 50 aligned with lane 154F is missing.

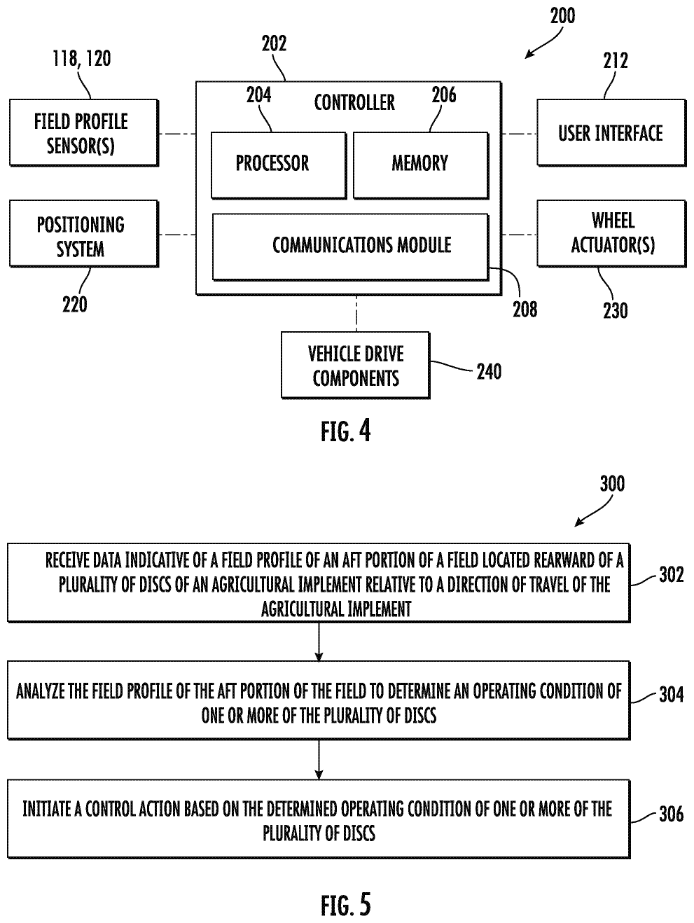

Referring now to FIG. 4, a schematic view of one embodiment of a system 200 for monitoring the operating condition of discs 50 of an agricultural implement is illustrated in accordance with aspects of the present subject matter. For purposes of discussion, the system 200 will be described herein with reference to the implement 10 described above and shown in FIG. 1 and the example field profiles described above and shown in FIGS. 2 and 3. However, it should be appreciated that the disclosed system 200 may generally be utilized with any suitable implement having any suitable implement configuration. Additionally, it should be appreciated that communicative links or electrical couplings of the system 200 shown in FIG. 4 are indicated by dashed lines.

As shown, the system 200 includes a controller 202 configured to electronically control the operation of one or more components of the agricultural implement 10 and/or the associated work vehicle 14 configured to tow the agricultural implement 10. In general, the controller 202 may comprise any suitable processor-based device known in the art, such as a computing device or any suitable combination of computing devices. Thus, in several embodiments, the controller 202 may include one or more processor(s) 204, and associated memory device(s) 206 configured to perform a variety of computer-implemented functions. As used herein, the term “processor” refers not only to integrated circuits referred to in the art as being included in a computer, but also refers to a controller, a microcontroller, a microcomputer, a programmable logic circuit (PLC), an application specific integrated circuit, and other programmable circuits. Additionally, the memory device(s) 206 of the controller 202 may generally comprise memory element(s) including, but not limited to, a computer readable medium (e.g., random access memory RAM)), a computer readable non-volatile medium (e.g., a flash memory), a floppy disk, a compact disc-read only memory (CD-ROM), a magneto-optical disk (MOD), a digital versatile disc (DVD) and/or other suitable memory elements. Such memory device(s) 206 may generally be configured to store suitable computer-readable instructions that, when implemented by the processor(s) 204, configure the controller 202 to perform various computer-implemented functions, such as one or more aspects of the methods and algorithms that will be described herein. In addition, the controller 202 may also include various other suitable components, such as a communications circuit or module, one or more input/output channels, a data/control bus and/or the like.

It should be appreciated that, in several embodiments, the controller 202 may correspond to an existing controller of the agricultural implement 10 and/or of the work vehicle 14 to which the implement 10 is coupled. However, it should be appreciated that, in other embodiments, the controller 202 may instead correspond to a separate processing device. For instance, in one embodiment, the controller 202 may form all or part of a separate plug-in module that may be installed within the agricultural implement 10 (and/or the associated work vehicle 14) to allow for the disclosed system and method to be implemented without requiring additional software to be uploaded onto existing control devices of the agricultural implement 10.

In some embodiments, the controller 202 may include a communications module or interface 208 to allow for the controller 202 to communicate with any of the various other system components described herein. For instance, the controller 202 may, in several embodiments, be configured to receive data from one or more sensors of the agricultural implement 10 that is used to detect one or more parameters associated with the operating condition of the discs 50 of the implement 10. Particularly, the controller 202 may be in communication with one or more field profile sensors 118, 120 (e.g., one or more surface profile sensors 118 and/or one or more seedbed profile sensors 120) configured to detect one or more parameters associated with or indicative of the field profile (e.g., the surface profile and/or the seedbed profile) at a location aft of the implement 10, which can be used to determine or infer the operating condition of the discs 50. In one embodiment, the controller 202 may be communicatively coupled to the field profile sensor(s) 118, 120 via any suitable connection, such as a wired or wireless connection, to allow data to be transmitted from the sensor(s) 118, 120 to the controller 202.

As indicated above, the field profile sensor(s) 118, 120 may be installed or otherwise positioned relative to the implement 10 to capture data (e.g., point-cloud data, image data, radar data, ultrasound data, and/or the like) indicative of the field profile of an aft portion of the field, which, in turn, is indicative of the operating condition of the discs 50, such as whether a given disc 50 is damaged or missing. Thus, in several embodiments, the controller 202 may be configured to monitor the operating condition of the discs 50 based on the data received from the sensor(s) 118, 120. For example, the controller 202 may be configured to analyze/process the received data to monitor the field profile detected across the aft portion of the field relative to an expected or baseline field profile. For instance, the controller 202 may include one or more suitable algorithms stored within its memory 206 that, when executed by the processor 204, allow the controller 202 to infer the operating condition of one or more discs 50 based on the comparison between the detected or measured field profile and the expected or baseline field profile of the field. Specifically, as indicated above, the shape or dimensions of the measured field profile within each lane 154 aligned with a given disc 50 may be compared to the shape or dimensions of the expected or baseline field profile to determine or infer the operating condition of the respective disc 50.

The controller 202 may also be configured to perform one or more control actions based on the determination of the operating condition of the discs 50. For instance, the controller 202 may be configured to indicate to an operator the operating condition of each disc 50, such as by indicating whether a given disc 50 is damaged or missing. For example, in the embodiment shown in FIG. 4, the communications module 208 may allow the controller 202 to communicate with a user interface 212 having a display device, with the display device being configured to display information associated with the operating condition of one or more of the discs 50. However, it should be appreciated that the controller 202 may instead be coupled to any number of other indicators, such as lights, alarms and/or the like to provide an indicator to the operator regarding the operating condition of the discs 50.

Is some embodiments, the controller 202 may further be configured to indicate the location within the field at which a given disc 50 becomes damaged or falls of the implement 10. In one embodiment, the controller 202 may indicate the location by mapping or georeferencing the location at which the controller 202 initially detects or determines that a disc 50 is damaged or missing. For example, as shown in FIG. 4, the controller 202 may be in communication with a positioning system 220 (e.g., a GPS-based positioning system), with the positioning system 220 being configured to identify the current location of the implement 10. In such an embodiment, the controller 202 may be configured to monitor the current location of the implement 10 as it simultaneously monitors the operating condition of each disc 50. When it is detected that a given disc 50 is damaged or missing, the controller 202 may store the current field location of the implement 10 within its memory 206. The controller 202 may then create an alert or log of alerts to indicate to an operator the identified location(s), which may, for example, be displayed to the operator via the user interface 212. For instance, a field map may be displayed to the operator that maps the location so that the operator can subsequently inspect the location for a cause of the damaged or missing disc (e.g., due to rocks or other obstacles within the field).

In further embodiments, the controller 202 may be configured to perform one or more implement-related control actions based on the determination of the operating condition of the discs 50. Specifically, in some embodiments, the controller 202 may be configured to control one or more components of the agricultural implement 10 based on the determination that one of the discs 50 is damaged or missing. For example, as shown in FIG. 4, the controller 202 may be configured to control one or more wheel actuators 230 (e.g., actuators 74, 76, 78, 80, 82, 84, 90, 92 of implement 10) to move the implement frame into its raised position when it is determined that one or more of the discs 50 is damaged or missing.

Additionally or alternatively, in some embodiments, the controller 202 may be configured to perform one or more vehicle-related control actions based on the determination of the operating condition of the discs 50. For example, as shown in FIG. 4, in some embodiments, the controller 202 may be configured to control the operation of one or more vehicle drive components 240 configured to drive the work vehicle coupled to the implement, such as the engine 15A and/or the transmission 15B of the work vehicle 14. In such embodiments, the controller 202 may be configured to control the operation of the vehicle drive component(s) 240 based on the determination of the operating condition of the discs 50, for example, to bring the work vehicle 14 and implement 10 to a stop when it is determined that one or more of the discs 50 is damaged or missing.

It should be appreciated that, depending on the type of controller 202 being used, the above-described control actions may be executed directly by the controller 202 or indirectly via communications with a separate controller. For instance, when the controller 202 corresponds to an implement controller of the implement 10, the controller 202 may be configured to execute the implement-related control actions directly while being configured to execute the vehicle-related control actions by transmitting suitable instructions or requests to a vehicle-based controller of the work vehicle towing the implement 10 (e.g., using an ISObus communications protocol). Similarly, when the controller 202 corresponds to a vehicle controller of the vehicle towing the implement 10, the controller 202 may be configured to execute the vehicle-related control actions directly while being configured to execute the implement-related control actions by transmitting suitable instructions or requests to an implement-based controller of the implement 10 (e.g., using an ISObus communications protocol). In other embodiments, the controller 202 may be configured to execute both the implement-based control actions and the vehicle-based control actions directly or the controller 202 may be configured to execute both of such control action types indirectly via communications with a separate controller.

Referring now to FIG. 5, a flow diagram of one embodiment of a method 300 for monitoring the operating condition of discs of agricultural implements is illustrated in accordance with aspects of the present subject matter. In general, the method 300 will be described herein with reference to the implement 10 shown in FIG. 1, the exemplary field profiles shown in FIGS. 2 and 3, and the system 200 shown in FIG. 4. However, it should be appreciated that the disclosed method 300 may be executed with implements having any other suitable configurations and/or with systems having any other suitable system configuration. In addition, although FIG. 5 depicts steps performed in a particular order for purposes of illustration and discussion, the methods discussed herein are not limited to any particular order or arrangement. One skilled in the art, using the disclosures provided herein, will appreciate that various steps of the methods disclosed herein can be omitted, rearranged, combined and/or adapted in various ways without deviating from the scope of the present disclosure.

As shown in FIG. 8, at (302), the method 300 may include receiving data indicative of a field profile of an aft portion of a field located rearward of a plurality of discs of an agricultural implement relative to a direction of travel of the agricultural implement. For instance, as indicated above, the controller 202 may receive data indicative of a field profile of the aft portion of the field rearward of the discs 50 of an agricultural implement 10 relative to a direction of travel 18 of the implement 10, such as by receiving such data from a surface profile sensor(s) 118 associated with the surface profile of the field and/or data from a seedbed profile sensor(s) 120 associated with the seedbed profile of the field.

Moreover, at (304), the method 300 may include analyzing the field profile of the aft portion of the field to determine an operating condition of one or more of the plurality of discs. For instance, as described above, the controller 202 may compare the measured field profile of the aft portion of the field to a baseline field profile to determine whether one or more discs 50 are damaged or missing.

Additionally, at (306), the method 300 may include initiating a control action based on the determined operating condition of one or more of the plurality of discs. For instance, as indicated above, in some embodiments, the controller 202 may provide an indication of the operating condition to the operator, such as by controlling the operation of the user interface 212 to display information indicating that one or more of the discs 50 is damaged or missing. In addition to such operator notifications or as an alternative thereto, the controller 202 may be configured to identify a location at which the damaged or missing disc 50 was initially detected, such as by mapping or georeferencing the location. Moreover, in some embodiments, the controller 202 may be configured to execute one or more implement-based or vehicle-based control actions, such as by controlling the operation of an actuator 230 of the implement 10 to adjust the penetration depth of the discs 50 or by bringing the implement 10 to a stop by controlling the operation of the associated work vehicle 14.

It is to be understood that the steps of the method 300 are performed by the controller 202 upon loading and executing software code or instructions which are tangibly stored on a tangible computer readable medium, such as on a magnetic medium, e.g., a computer hard drive, an optical medium, e.g., an optical disc, solid-state memory, e.g., flash memory, or other storage media known in the art. Thus, any of the functionality performed by the controller 202 described herein, such as the method 300, is implemented in software code or instructions which are tangibly stored on a tangible computer readable medium. The controller 202 loads the software code or instructions via a direct interface with the computer readable medium or via a wired and/or wireless network. Upon loading and executing such software code or instructions by the controller 202, the controller 202 may perform any of the functionality of the controller 202 described herein, including any steps of the method 300 described herein.

The term “software code” or “code” used herein refers to any instructions or set of instructions that influence the operation of a computer or controller. They may exist in a computer-executable form, such as machine code, which is the set of instructions and data directly executed by a computer's central processing unit or by a controller, a human-understandable form, such as source code, which may be compiled in order to be executed by a computer's central processing unit or by a controller, or an intermediate form, such as object code, which is produced by a compiler. As used herein, the term “software code” or “code” also includes any human-understandable computer instructions or set of instructions, e.g., a script, that may be executed on the fly with the aid of an interpreter executed by a computer's central processing unit or by a controller.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. A system for monitoring the condition of discs of agricultural implements, the system comprising:
a plurality of discs configured to be supported relative to an agricultural implement;
a field profile sensor configured to generate data indicative of a field profile of an aft portion of the field located rearward of the plurality of discs relative to a direction of travel of the agricultural implement; and
a controller communicatively coupled to the field profile sensor, the controller being configured to monitor the data received from the field profile sensor and determine an operating condition of one or more of the plurality of discs based at least in part on the field profile of the aft portion of the field.

2. The system of claim 1, wherein field profile sensor comprises a surface profile sensor and the field profile comprises a surface profile of the aft portion of the field.

3. The system of claim 2, wherein the surface profile sensor comprises one of a LIDAR device, a camera, a radar sensor or an ultrasound sensor.

4. The system of claim 1, wherein field profile sensor comprises a seedbed profile sensor and the field profile comprises a seedbed profile of the aft portion of the field.

5. The system of claim 4, wherein the seedbed profile sensor comprises a ground-penetrating radar or an electromagnetic induction device.

6. The system of claim 1, wherein the controller is configured to determine the operating condition of the one or more of the plurality of discs by comparing the field profile of the aft portion of the field to a baseline field profile.

7. The system of claim 1, wherein the operating condition comprises a damaged condition and wherein the controller is configured to determine that a disc of the plurality of discs is experiencing the damaged condition when data received from the field profile sensor indicates a cyclical deviation in the field profile relative to a baseline field profile for a section of the field aligned with the disc in the direction of travel of the agricultural implement.

8. The system of claim 1, wherein the operating condition comprises a missing condition and wherein the controller is configured to determine that a disc of the plurality of discs is missing when data received from the field profile sensor indicates a constant deviation in the field profile relative to a baseline field profile for a section of the field aligned with the disc in the direction of travel of the agricultural implement.

9. The system of claim 1, wherein the controller is further configured to initiate a control action based on the determined operating condition of the one or more of the plurality of discs.

10. The system of claim 9, wherein the control action comprises generating an operator notification associated with the operating condition of the one or more of the plurality of discs or adjusting an operation of at least one of the agricultural implement or a work vehicle towing the agricultural implement.

11. A method for monitoring the condition of discs of agricultural implements, the method comprising:
receiving, with a computing device, data indicative of a field profile of an aft portion of a field located rearward of a plurality of discs of an agricultural implement relative to a direction of travel of the agricultural implement;
analyzing, with the computing device, the field profile of the aft portion of the field to determine an operating condition of one or more of the plurality of discs; and
initiating, with the computing device, a control action based on the determined operating condition of one or more of the plurality of discs.

12. The method of claim 11, wherein receiving data indicative of the field profile of the aft portion of the field comprises receiving data from a surface profile sensor indicative of a surface profile of the aft portion of the field.

13. The method of claim 12, wherein the surface profile sensor comprises one of a LIDAR device, a camera, a radar sensor or an ultrasound sensor.

14. The method of claim 11, wherein receiving data indicative of the field profile of the aft portion of the field comprises receiving data from a seedbed profile sensor indicative of a seedbed profile of the aft portion of the field.

15. The method of claim 14, wherein the seedbed profile sensor comprises a ground-penetrating radar or an electromagnetic induction device.

16. The method of claim 11, wherein analyzing the field profile of the aft portion of the field to determine the operating condition of the one or more of the plurality of discs comprises comparing the field profile of the aft portion of the field to a baseline field profile.

17. The method of claim 11, wherein the operating condition comprises a damaged condition and wherein analyzing the field profile of the aft portion of the field to determine the operating condition of the one or more of the plurality of discs comprises determining that a disc of the plurality of discs is experiencing the damaged condition when data received from the field profile sensor indicates a cyclical deviation in the field profile relative to a baseline field profile for a section of the field aligned with the disc in the direction of travel of the agricultural implement.

18. The method of claim 11, wherein the operating condition comprises a missing condition and wherein analyzing the field profile of the aft portion of the field to determine the operating condition of the one or more of the plurality of discs comprises determining that a disc of the plurality of discs is missing when data received from the field profile sensor indicates a constant deviation in the field profile relative to a baseline field profile for a section of the field aligned with the disc in the direction of travel of the agricultural implement.

19. The method of claim 11, wherein initiating the control action comprises generating an operator notification associated with the operating condition of the one or more of the plurality of discs.

20. The method of claim 11, wherein initiating the control action comprises adjusting an operation of at least one of the agricultural implement or a work vehicle towing the agricultural implement.
